(19) United States
(12) Patent Application Publication
(10) Pub. No.: US 2013/0189186 A1
(43) Pub. Date: Jul. 25, 2013

(54) APOPTOSIS IMAGING AGENTS BASED ON LANTIBIOTIC PEPTIDES

(71) Applicants: Bard Indrevoll, Oslo (NO); Duncan Hiscock, Amersham (GB); Bente Elizabeth Arbo, Oslo (NO); Rajiv Bhalla, Amersham (GB); Matthias Eberhard Glaser, London (GB); Graeme Walter McRobbie, Amersham (GB)

(72) Inventors: Bard Indrevoll, Oslo (NO); Duncan Hiscock, Amersham (GB); Bente Elizabeth Arbo, Oslo (NO); Rajiv Bhalla, Amersham (GB); Matthias Eberhard Glaser, London (GB); Graeme Walter McRobbie, Amersham (GB)

(73) Assignee: GE HEALTHCARE LIMITED, LITTLE CHALFONT, BUCKINGHAMSIRE (GB)

(21) Appl. No.: 13/876,187

(22) PCT Filed: Sep. 27, 2011
(86) PCT No.: PCT/EP2011/066789

§ 371 Date: Mar. 27, 2013

(30) Foreign Application Priority Data
Sep. 27, 2010 (GB) 1016206.3

(51) Int. Cl. A61K 51/08

(52) U.S. Cl. CPC A61K 51/088
USPC 424/1.69; 530/317

(57) ABSTRACT

The present invention relates to radiopharmaceutical imaging in vivo of apoptosis. The invention provides imaging agents which target apoptotic cells via selective binding to the aminophospholipid phosphatidylethanolamine (PE), which is exposed on the surface of apoptotic cells. The radiopharmaceuticals comprise radiometal complexes of chelator conjugates of PE-binding peptides. Also provided are pharmaceutical compositions, kits and methods of in vivo imaging.

### FIELD OF THE INVENTION

The present invention relates to radiopharmaceutical imaging in vivo of apoptosis and other forms of cell death. The invention provides imaging agents which target apoptotic cells via selective binding to the aminophospholipid phosphatidylethanolamine (PE), which is exposed on the surface of apoptotic cells. Also provided are pharmaceutical compositions, kits and methods of in vivo imaging.

### BACKGROUND TO THE INVENTION

Apoptosis or programmed cell death (PCD) is the most prevalent cell death pathway and proceeds via a highly regulated, energy-conserved mechanism. In the healthy state, apoptosis plays a pivotal role in controlling cell growth, regulating cell number, facilitating morphogenesis, and removing harmful or abnormal cells. Dysregulation of the PCD process has been implicated in a number of disease states, including those associated with the inhibition of apoptosis, such as cancer and autoimmune disorders, and those associated with hyperactive apoptosis, including neurodegenerative diseases, haematologic diseases, AIDS, ischaemia and allograft rejection. The visualization and quantitation of apoptosis is therefore useful in the diagnosis of such apoptosis-related pathophysiology.

Therapeutic treatments for these diseases aim to restore balanced apoptosis, either by stimulating or inhibiting the PCD process as appropriate. Non-invasive imaging of apoptosis in cells and tissue in vivo is therefore of immense value for early assessment of a response to therapeutic intervention, and can provide new insight into devastating pathological processes. Of particular interest is early monitoring of the efficacy of cancer therapy to ensure that malignant growth is controlled before the condition becomes terminal.

There has consequently been great interest in developing imaging agents for apoptosis [see eg. Zeng et al, Anti-cancer Agent Med. Chem., 9(9), 986-995 (2009); Zhao, ibid, 9(9), 1018-1023 (2009) and M. De Saint-Hubert et al, Methods, 48, 178-187 (2009)]. Of the probes available for imaging cell death, radiolabelled Annexin V has received the most attention. Annexin V binds only to negatively charged phospholipids, which renders it unable to distinguish between apoptosis and necrosis.

The lanthionine-containing antibiotic peptides (“lantibiotics”) duramycin and cinnamycin are two closely related 19-mer peptides with a compact tetracyclic structure [Zhao, Amino Acids, DOI 10.1007/s00726-009-0386-9, Springer-Verlag (2009), and references cited therein]. They are crosslinked via four covalent, intramolecular bridges, and differ by only a single amino acid residue at position 2. The structures of duramycin and cinnamycin are shown schematically below, where the numbering refers to the position of the linked amino acid residues in the 19-mer sequence:

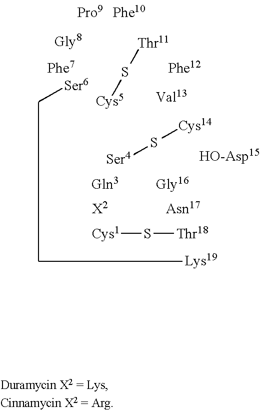

Programmed cell death or apoptosis is an intracellular, energy-dependent self-destruction of the cell. The redistribution of phospholipids across the bilayer of the cell plasma membrane is an important marker for apoptosis. Thus, in viable cells, the aminophospholipids phosphatidylethanolamine (PE or PtdE) and phosphatidylserine (PS) are predominantly constituents of the inner leaflet of the cell plasma membrane. In apoptopic cells, there is a synchronised externalization of PE and PS.

Both duramycin and cinnamycin bind to the neutral aminophospholipid PE with similar specificity and high affinity, by forming a hydrophobic pocket that fits around the PE head-group. The binding is stabilised by ionic interaction between the β-hydroxyaspartic acid residue (HO-Asp¹⁵) and the ethanolamine group. Modifications to this residue are known to inactivate duramycin [Zhao et al, J. Nucl. Med, 49, 1345-1352 (2008)]. Zhao [Amino Acids, DOI 10.1007/s00726-009-0386-9, Springer-Verlag (2009)] cites earlier work by Wakamatsu et al [Biochemistry, 29, 113-188 (1990)], where NMR studies show that none of the ¹H NMR resonances of the 5 terminal amino acids of cinnamycin are shifted on binding to PE—suggesting that they are not involved in interactions with PE.

US 2004/0147440 A1 (University of Texas System) describes labelled anti-aminophospholipid antibodies, which can be used to detect pre-apoptopic or apoptopic cells, or in cancer imaging. Also provided are conjugates of duramycin with biotin, proteins or anti-viral drugs for cancer therapy.

WO 2006/055855 discloses methods of imaging apoptosis using a radiolabelled compound which comprises a phosphatidylserine-binding C2 domain of a protein.

WO 2009/114549 discloses a radiopharmaceutical made by a process comprising:
(i) providing a polypeptide having at least 70% sequence similarity with CKQSCSFGPFTFVCDGNTK,
   wherein the polypeptide comprises a thioether bond between amino acids residues 1-18, 4-14, and 5-11, and an amide bond between amino acids residues 6-19, and, wherein one or more distal moieties of structure

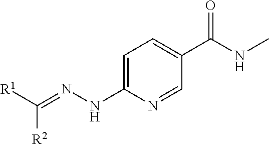

are covalently bound to the amino acid at position 1, position 2, or, positions 1 and 2 of the polypeptide, and wherein R¹ and R² are each independently a straight or branched, saturated or unsaturated C₁₋₄ alkyl; and
(ii) chelating one or more of the distal moieties with ^{99m}Tc^{x}, (^{99m}Tc═O)³⁺, (^{99m}Tc≡N)²⁺, (O═^{99m}Tc═O)⁺ or [^{99m}Tc(CO)₃]⁺, wherein x is a redox or oxidation state selected from the group consisting of +7, +6, +5, +4, +3, +2, +1, 0 and −1, or, a salt, solvate or hydrate thereof.

The ‘distal moiety’ of WO 2009/114549 is a complexing agent for the radioisotope ^{99m}Tc, which is based on hydrazinonicotinamide (commonly abbreviated “HYNIC”). HYNIC is well known in the literature [see e.g. Banerjee et al, Nucl. Med. Biol, 32, 1-20 (2005)], and is a preferred method of labelling peptides and proteins with ^{99m}Tc [R. Alberto, Chapter 2, pages 19-40 in IAEA Radioisotopes and Radiopharmaceuticals Series 1: “Technetium-99m Radiopharmaceuticals Status and Trends” (2009)].

WO 2009/114549 discloses specifically ^{99m}Tc-HYNIC-duramycin, and suggests that the radiopharmaceuticals taught therein are useful for imaging apoptosis and/or necrosis, atherosclerotic plaque or acute myocardial infarct.

Zhao et al [J. Nucl. Med, 49, 1345-1352 (2008)] disclose the preparation of ^{99m}Tc-HYNIC-duramycin. Zhao et al note that duramycin has 2 amine groups available for conjugation to HYNIC: at the N-terminus (Cys¹ residue), and the epsilon-amine side chain of the Lys² residue. They purified the HYNIC-duramycin conjugate by HPLC to remove the bis-HYNIC-functionalised duramycin, prior to radiolabelling with ^{99m}Tc. Zhao et al acknowledge that the ^{99m}Tc-labelled mono-HYNIC-duramycin conjugates studied are probably in the form of a mixture of isomers.

Whilst HYNIC forms stable ^{99m}Tc complexes, it requires additional co-ligands to complete the coordination sphere of the technetium metal complex. The HYNIC may function as a monodentate ligand or as a bidentate chelator depending on the nature of the amino acid side chain functional groups in the vicinity [King et al, Dalton Trans., 4998-5007 (2007); Meszaros et al [Inorg. Chim. Acta, 363, 1059-1069 (2010)]. Thus, depending on the environment, HYNIC forms metal complexes having 1- or 2-metal donor atoms. Meszaros et al note that the nature of the co-ligands used with HYNIC can have a significant effect on the behaviour of the system, and state that none of the co-ligands is ideal.

### THE PRESENT INVENTION

The present invention provides radiopharmaceutical imaging agents, particularly for imaging disease states of the mammalian body where abnormal apoptosis is involved. The imaging agents comprise radiometal chelator conjugates of a lantibiotic peptide. The invention provides radiometal complexes which form reproducibly, in high radiochemical purity (RCP), without the need for co-ligands. The present inventors have also established that attachment of the radiometal complex at the N-terminus (Cys^{a} residue) of the lantibiotic peptide of Formula II herein is strongly preferred, since attachment of the uncomplexed chelator at even the amino acid adjacent to the N-terminus (Xaa of Formula II) has a deleterious effect on binding to phosphatidylethanolamine. This effect was not recognized previously in the prior art, and hence the degree of impact on binding affinity is believed novel.

### DETAILED DESCRIPTION OF THE INVENTION

In a first aspect, the present invention provides an imaging agent which comprises a compound of Formula I:

Z¹-(L)ₙ-[LBP]-Z² (I)
wherein:
LBP is a lantibiotic peptide of Formula II:

Cys^{a}-Xaa-Gln-Ser^{b}-Cys^{c}-Ser^{d}-Phe-Gly-Pro-Phe-Thr^{c}-Phe-Val-Cys^{b}-(HO-Asp)-Gly-Asn-Thr^{a}-Lys^{d} (II)
Xaa is Arg or Lys;
Cys^{a}-Thr^{a}, Ser^{b}-Cys^{b} and Cys^{c}-Thr^{c} are covalently linked via thioether bonds;
Ser^{d}-Lys^{d} are covalently linked via a lysinoalanine bond;
HO-Asp is β-hydroxyaspartic acid;
Z¹-(L)ₙ- is attached to Cys^{a} and optionally also to Xaa of LBP, wherein Z¹ comprises a radiometal complex of a chelating agent having at least 4 metal donor atoms;
Z² is attached to the C-terminus of LBP and is OH, OB^{c}, or M^{IG},
   where B^{C} is a biocompatible cation; and
   M^{IG} is a metabolism inhibiting group which is a biocompatible group which inhibits or suppresses in vivo metabolism of the LBP peptide;
L is a synthetic linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1.

By the term “imaging agent” is meant a compound suitable for imaging the mammalian body. Preferably, the mammal is an intact mammalian body in vivo, and is more preferably a human subject. Preferably, the imaging agent can be administered to the mammalian body in a minimally invasive manner, i.e. without a substantial health risk to the mammalian subject when carried out under professional medical expertise. Such minimally invasive administration is preferably intravenous administration into a peripheral vein of said subject, without the need for local or general anaesthetic. The imaging agents of the first aspect are particularly suitable for imaging apoptosis and other forms of cell death, as is described in the sixth aspect (below).

The term “in vivo imaging” as used herein refers to those techniques that non-invasively produce images of all or part of an internal aspect of a mammalian subject.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure.

By the term “peptide” is meant a compound comprising two or more amino acids, as defined above, linked by a peptide bond (i.e. an amide bond linking the amine of one amino acid to the carboxyl of another).

The term “lantibiotic peptide” refers to a peptide containing at least one lanthionine bond. “Lanthionine” has its conventional meaning, and refers to the sulfide analogue of cystine, having the chemical structure shown:

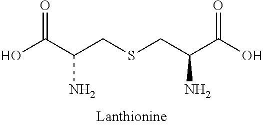 [SMILES: N[C@@H](CSC[C@H](N)C(=O)O)C(=O)O]

By the term “covalently linked via thioether bonds” is meant that the thiol functional group of the relevant Cys residue is linked as a thioether bond to the Ser or Thr residue shown via dehydration of the hydroxyl functional group of the Ser or Thr residue, to give lanthionine or methyllanthionine linkages. Such linkages are described by Willey et al [Ann. Rev. Microbiol., 61, 477-501 (2007)].

By the term “lysinoalanine bond” is meant that the epsilon amine group of the Lys residue is linked as an amine bond to the Ser residue shown via dehydration of the hydroxyl functional group of the Ser giving a —(CH₂)—NH—(CH₂)₄— linkage joining the two alpha-carbon atoms of the amino acid residues.

By the term “radiometal complex” is meant a coordination metal complex of the radiometal with the chelator, wherein said chelator is covalently bonded to the LBP peptide via the linker group (L) of Formula I. The coordination complex does not comprise hydrazinonicotinamide (HYNIC) ligands bound to the radiometal. Hence, the chelator is the principal species binding to the radiometal—it is not simply a co-ligand for HYNIC.

The term “chelating agent” has its conventional meaning and refers to 2 or more metal donor atoms arranged such that chelate rings, preferably 5- to 7-membered chelate rings, result upon metal coordination, more preferably 5- or 6-membered chelate rings. The metal donor atoms are covalently linked by a non-coordinating backbone of either carbon atoms or non-coordinating heteroatoms. The chelating agent can be macrocyclic or open chain. The chelating agents of the present invention comprise at least 4 metal donor atoms, suitably 4 to 8 metal donor atoms, in which at least 4 such metal donor atoms are bound to the radiometal in the radiometal complex.

Suitable radiometals of the present invention include: ^{99m}Tc, ^{94m}Tc, ¹⁸⁶Re, ¹⁸⁸Re, ⁶⁴Cu, ⁶⁷Cu, ⁶⁷Ga, ⁶⁸Ga, ¹⁰⁵Rh, ^{101m}Rh, ¹¹¹In, ⁸⁹Zr or ⁴⁵Ti.

When Z¹ is attached to Cys^{a}, it is attached to the N-terminus of the LBP peptide. When Z¹ is also attached to Xaa, that means that Xaa is Lys, and Z¹ is attached to the epsilon amino group of the Lys residue.

The Z² group substitutes the carbonyl group of the last amino acid residue of the LBP—i.e. the carboxy terminus. Thus, when Z² is OH, the carboxy terminus of the LBP terminates in the free CO₂H group of the last amino acid residue, and when Z² is OB^{c} that terminal carboxy group is ionised as a CO₂B^{c} group.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “metabolism inhibiting group” (M^{IG}) is meant a biocompatible group which inhibits or suppresses in vivo metabolism of the LBP peptide at the carboxy terminus (Z²). Such groups are well known to those skilled in the art and are suitably chosen from: carboxamide, tert-butyl ester, benzyl ester, cyclohexyl ester, amino alcohol or a polyethyleneglycol (PEG) building block. The LBP peptides of the invention are known to exhibit high in vivo metabolic stability (95% at 60 min), hence Z² is preferably OH or OB^{c}.

### PREFERRED EMBODIMENTS

The chelating agent is preferably designed such that the chelate rings formed on complexation with the radiometal comprise at least one 5- or 6-membered ring, more preferably 2 to 4 such rings, most preferably 3 or 4 such rings.

The chelating agent is preferably chosen from: an aminocarboxylate ligand having at least 6 donor atoms; or a tetradentate chelator having an N₃S, N2S2 or N4 donor set. The chelating agent is more preferably either an aminocarboxylate ligand having at least 6 donor atoms, or a tetradentate chelator having an N4 donor set, and most preferably a tetradentate chelator having an N4 donor set.

The term “aminocarboxylate ligand” has its conventional meaning, and refers to a chelating agent of the EDTA, DTPA type. The donor atoms of such chelators are a mixture of amine (N) donors and carboxylic acid (O) donors. Such chelators may be open chain (e.g. EDTA, DTPA or HBED), or macrocyclic (eg. DOTA or NOTA). Suitable such chelators include DOTA, HBED and NOTA, which are well known in the art and are preferred for radiometals such as ⁶⁷Ga or ⁶⁸Ga, ¹¹¹In, radioisotopes of copper, ⁸⁹Zr and ⁴⁵Ti.

The term “tetradentate chelator” has its conventional meaning and refers to a chelating agent in which the radiometal is coordinated by the four metal donor atoms of the tetradentate chelating agent.

By the term “N3 S donor set” is meant that the four metal donor atoms of the tetradentate chelator are made up of 3 nitrogen donor atoms and one sulfur donor atom. Examples of suitable such N donor atom types are: amines (especially primary or secondary amines); amides or oximes, or combinations thereof. Examples of suitable such S donor atom types are: thiol and thioether. Preferred such N3S chelators have a thioltriamide donor set, and are preferably open chain chelators such as MAG3 (mercaptoacetyltriglycine).

By the term “N2S2 donor set” is meant that the four metal donor atoms of the tetradentate chelator are made up of 2 nitrogen donor atoms and 2 sulfur donor atoms. Suitable N and S donor atoms are as described for N3 S (above). Preferred such N2S2 chelators have a diaminedithiol or amideaminedithiol donor set, and are preferably open chain chelators such as BAT or N,N-ethylenedi-L-cysteine [Inorg Chem., 35(2):404-414 (1996)].

By the term “N4 donor set” is meant that the four metal donor atoms of the tetradentate chelator are all based on nitrogen. Examples of suitable such N donor atom types are: amines (especially primary or secondary amines); amides or oximes, or combinations thereof.

The N4 donor set is preferably chosen from: diaminedioxime; tetra-amine; amidetriamine, or diamidediamine. The N4 chelator can be open-chain or macrocyclic (eg. cyclam, cyclen, monoxocyclam or dioxocyclam). Preferred N4 tetradentate chelating agents of the present invention have a diaminedioxime or a tetra-amine donor set, and are more preferably open-chain diaminedioximes or open-chain tetra-amines.

Preferred diaminedioxime chelators are of formula:

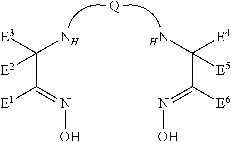

where E¹-E⁶ are each independently an R′ group;
each R′ is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring;
and Q is a bridging group of formula -(J)_{f}-;
where f is 3, 4 or 5 and each J is independently —O—, —NR′— or —C(R′)₂— provided that -(J)_{f}- may contain a maximum of one J group which is —O— or —NR′—.

Preferred Q groups are as follows:

Q=-(CH₂)(CHR′)(CH₂)— i.e. propyleneamine oxime or PnAO derivatives;
Q=-(CH₂)₂(CHR′)(CH₂)₂— i.e. pentyleneamine oxime or PentAO derivatives;

### Q=-(CH₂)₂NR′(CH₂)₂—.

E¹ to E⁶ are preferably chosen from: C₁₋₃ alkyl, C₂₋₄ alkoxyalkyl, C₁₋₃ hydroxyalkyl, C₁₋₃ fluoroalkyl, C₂₋₆ carboxyalkyl or C₁₋₃ aminoalkyl. Most preferably, each E¹ to E⁶ group is CH₃.
Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred diaminedioxime chelator has the Formula:

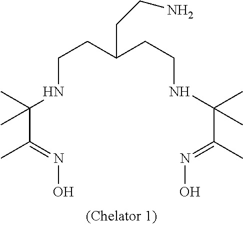 [SMILES: C/C(=N\O)C(C)(C)NCCC(CCN)CCNC(C)(C)/C(C)=N/O]

wherein the bridgehead primary amine group is conjugated to (L)ₙ (i.e. the linker group) and/or LBP peptide.

Preferred tetra-amine chelators are of formula:

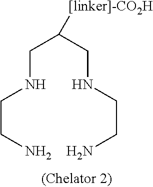

wherein the bridgehead carboxyl group is conjugated to the linker group and/or LBP peptide.

In Chelator 2, the [linker] is preferably a group of formula (A′)ₘ₁, where m1 is an integer of value 0 to 6, and each A′ is independently CH₂ or p-phenylene, where no more than one of the A′ groups is or p-phenylene. Preferably, each of the A′ groups is CH₂ and m1 is 1 to 6. A preferred such chelator is Chelator 2A, where the [linker] is —(CH₂)—.

The radiometal of the imaging agent is preferably ^{94m}Tc or ^{99m}Tc, and is more preferably ^{99m}Tc. For these technetium radioisotopes, the chelator is preferably a tetradentate with an N4 donor set as defined above.

Z² is preferably OH or OB^{c}.

In the imaging agent of the first aspect, Z¹ is preferably attached only to Cys^{a} of LBP. When Xaa is Arg, that means that Z¹ is attached to the LBP N-terminus, at the free amino group of the Cys^{a} residue. When Xaa is Lys, that means that steps are taken to either:
(i) selectively functionalise the LBP peptide at the Cys^{a} residue in preference to the epsilon amine group of the Xaa residue; or
(ii) a composition comprising LBP functionalized with Z¹ at both Cys^{a} and Xaa is prepared, then the Xaa-functionalised species is removed.

In the imaging agent of the first aspect, Xaa is preferably Arg.

The imaging agent of the first aspect preferably comprises a Linker Group (L), i.e. n in Formula (I) is preferably 1. L preferably comprises a PEG group of formula —(OCH₂CH₂)ₓ— where x is an integer of value 6 to 18, preferably 8 to 14, more preferably 10 to 12. Such linker groups are advantageous in reducing liver background retention and increasing urinary excretion of the imaging agent in vivo Preferably, L comprises a biomodifier group of Formula IA or IB:

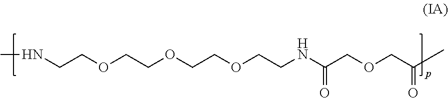

17-amino-5-oxo-6-aza-3,9,12,15-tetraoxaheptadecanoic acid of Formula IA wherein p is an integer from 1 to 10. In Formula IA, p is preferably 1, 2 or 3. Alternatively, a PEG-like structure based on a propionic acid derivative of Formula IB can be used:

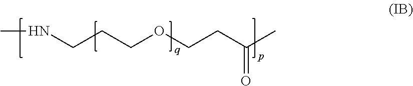

where p is as defined for Formula IA and q is an integer from 3 to 15.

In Formula IB, p is preferably 1 or 2, more preferably 1, and q is preferably 5 to 12, more preferably 12.

By the term “biomodifier” is meant a group which has an effect on the biodistribution of the agent in vivo.

The imaging agents of the first aspect can be obtained as described in the third aspect.

In a second aspect, the present invention provides a chelator conjugate of Formula III:

Z³-(L)ₙ-[LBP]-Z² (III)
wherein:
Z³ is a chelating agent having at least 4 metal donor atoms; and
L, n, LBP and Z² are as defined in the first aspect.

Preferred aspects of L, n, LBP and Z² and the chelating agent (Z³) in the second aspect are as defined in the first aspect (above).

Certain LBP peptides are commercially available. Thus, cinnamycin and duramycin are available from Sigma-Aldrich. Duramycin is produced by the strain: D3168 Duramycin from Streptoverticillium cinnamoneus. Cinnamycin can be biochemically produced by several strains, eg. from Streptomyces cinnamoneus or from Streptoverticillium griseoverticillatum. See the review by C. Chatterjee et al [Chem. Rev., 105, 633-683 (2005)]. Other peptides can be obtained by solid phase peptide synthesis as described in P. Lloyd-Williams, F. Albericio and E. Girald; Chemical Approaches to the Synthesis of Peptides and Proteins, CRC Press, 1997.

The chelator conjugates of the second aspect can be obtained as follows. When the chelator is a diaminedioxime, by reaction of the appropriate diamine with either:
(i) the appropriate chloronitroso derivative Cl—C(R¹)₂—CH(NO)R¹;
(ii) an alpha-chloro oxime of formula Cl—C(R¹)₂—C(═NOH)R¹;
(iii) an alpha-bromoketone of formula Br—C(R¹)₂—C(═O)R¹ followed by conversion of the diaminediketone product to the diaminedioxime with hydroxylamine.

Route (i) is described by S. Jurisson et al [Inorg. Chem., 26, 3576-82 (1987)]. Chloronitroso compounds can be obtained by treatment of the appropriate alkene with nitrosyl chloride (NOCl) as is known in the art. Further synthetic details of chloronitroso compounds are given by: Ramalingam [Synth. Commun., 25(5), 743-752 (1995)]; Glaser [J. Org. Chem., 61(3), 1047-48 (1996)]; Clapp [J. Org. Chem., 36(8) 1169-70 (1971)]; Saito [Shizen Kagaku, 47, 41-49 (1995)] and Schulz [Z. Chem., 21(11), 404-405 (1981)] Route (iii) is described in broad terms by Nowotnik et al [Tetrahedron, 50(29), p. 8617-8632 (1994)]. Alpha-chloro-oximes can be obtained by oximation of the corresponding alpha-chloro-ketone or aldehyde, which are commercially available. Alpha-bromoketones are commercially available.

More preferred tetra-amine chelators are of formula:

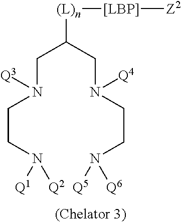

where:
   L, LBP, n and Z² are as defined in the first aspect;
   Q¹ to Q⁶ are independently Q groups, where Q is H or an amine protecting group.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). In some instances, the nature of the protecting group may be such that both the Q¹/Q² or Q⁵/Q⁶ groups, i.e. there is no NH bond on the associated amine nitrogen atom. The use of further protecting groups are described in ‘Protective Groups in Organic Synthesis’, 4^{th} Edition, Theorodora W. Greene and Peter G. M. Wuts, [Wiley Blackwell, (2006)]. Preferred amine protecting groups are Boc and Fmoc, most preferably Boc. When Boc is used, Q¹ and Q⁶ are both H, and Q², Q³, Q⁴ and Q⁵ are each tert-butoxycarbonyl.

Preferred aspects of L, LBP, n and Z² in Chelator 3 are as defined in the first aspect (above). Preferred Chelator 3 chelators have (L)ₙ=(A′)ₘ₁, where A′ and m1 and preferred aspects thereof are as described for Chelator 2 (above).

Tetra-amine chelators can be obtained as described in Scheme 1 (below). Further synthetic information on amino- and carboxy-functionalised tetra-amine chelators is provided by Abiraj et al [Chem. Eur. J., 16, 2115-2124 (2010)]. The synthesis of the Boc-protected tetra-amine analogue with a —(CH₂)₅OH bridgehead substituent has been described by Turpin et al [J. Lab. Comp. Radiopharm., 45, 379-393 (2002)]. The conjugation of tetra-amine chelators to biological targeting peptides is described by Nock et al [Eur. J. Nucl. Med., 30(2), 247-258 (2003)], and Maina et al [Eur. J. Nucl. Med., 30(9), 1211-1219 (2003)]. A bifunctional HBED derivative having a pendant active ester group is taught by Eder et al [Eur. J. Nucl. Med. Mol. Imaging, 35, 1878-1886 (2008)].

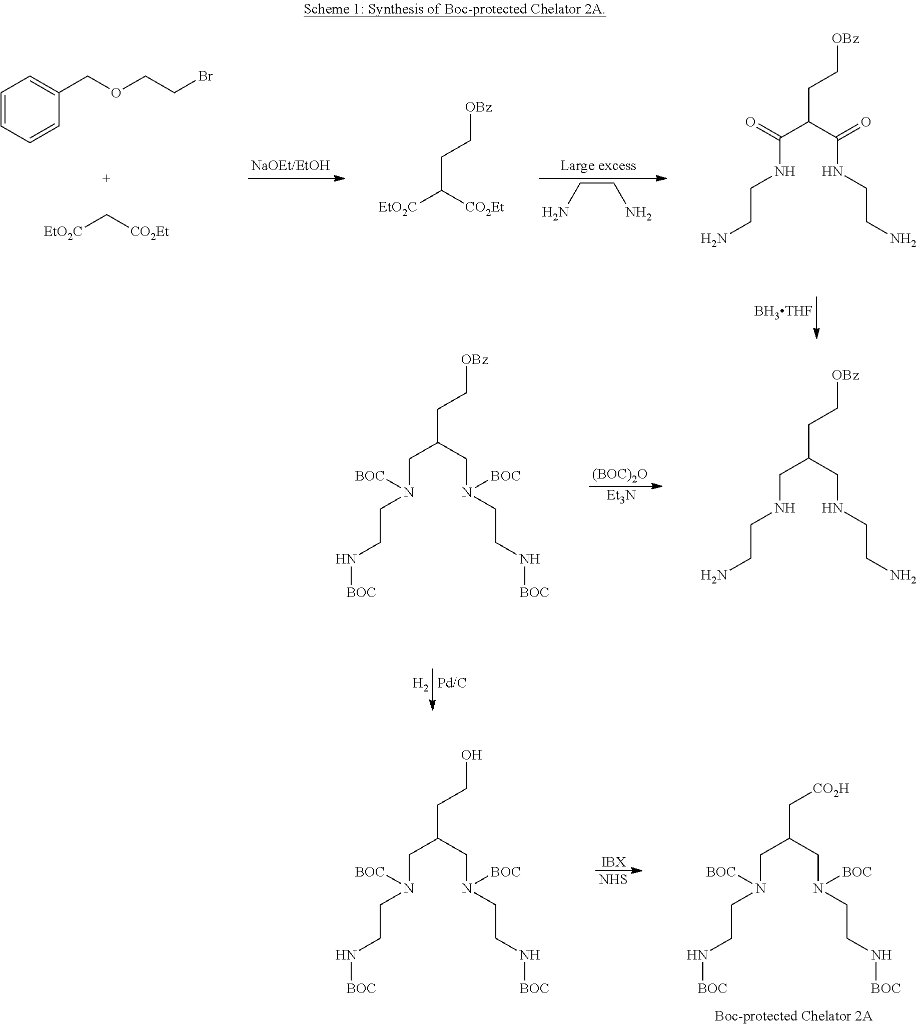

N3 S bifunctional chelators can be prepared by the method of Sudhaker et al [Bioconj. Chem., Vol. 9, 108-117 (1998)]. N₂S₂ Diamidedithiol compounds can be prepared by the method of Kung et al [Tetr. Lett., Vol 30, 4069-4072 (1989].

Monoamidemonoaminebisthiol compounds can be prepared by the method of Hansen et al [Inorg. Chem., Vol 38, 5351-5358 (1999)].

In a third aspect, the present invention provides a method of preparation of the imaging agent of the first aspect, which comprises reaction of the chelator conjugate of the second aspect with a supply of the desired radiometal in a suitable solvent.

Preferred aspects of the chelator conjugate and the radiometal in the third aspect are as described in the first and second aspects of the present invention (above).

The suitable solvent is typically aqueous in nature, and is preferably a biocompatible carrier solvent as defined in the fourth aspect (below).

In a fourth aspect, the present invention provides a radiopharmaceutical composition which comprises the imaging agent of the first aspect, together with a biocompatible carrier, in a form suitable for mammalian administration.

Preferred aspects of the imaging agent in the fourth aspect are as described in the first aspect of the present invention (above).

The “biocompatible carrier” is a fluid, especially a liquid, in which the imaging agent can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection, isotonic saline or phosphate buffer.

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (eg. blood). Such compositions also contain only biologically compatible excipients, and are preferably isotonic.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may contain additional optional excipients such as: an antimicrobial preservative, pH-adjusting agent, filler, radioprotectant, solubiliser or osmolality adjusting agent. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation as described above. By the term “solubiliser” is meant an additive present in the composition which increases the solubility of the imaging agent in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates; poly(oxyethylene)poly(oxypropylene)poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the second aspect may be prepared under aseptic manufacture (i.e. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

As noted above, the pharmaceutical compositions of the present invention preferably comprise a solubiliser, so that a sterile filtration step may be used without undue loss of radioactivity adsorbed to the filter material. Similar considerations apply to manipulations of the pharmaceutical compositions in clinical grade syringes, or using plastic tubing, where adsorption may cause loss of radioactivity without the use of a solubiliser.

The radiopharmaceutical compositions of the present invention may be prepared by various methods:
(i) aseptic manufacture techniques in which the radiometal complex formation is carried out in a clean room environment;
(ii) terminal sterilisation, in which the radiometal complex formation is carried out without using aseptic manufacture and then sterilised at the last step [eg. by gamma irradiation, autoclaving dry heat or chemical treatment (e.g. with ethylene oxide)];
(iii) kit methodology in which a sterile, non-radioactive kit formulation comprising the chelator conjugate of Formula III and optional excipients is reacted with a supply of the desired radiometal.

Method (iii) is preferred, and kits for use in this method are described in the fifth embodiment (below).

In a fifth aspect, the present invention provides a kit for the preparation of the radiopharmaceutical composition of the fourth aspect, which comprises the chelator conjugate of the second aspect in sterile, solid form such that upon reconstitution with a sterile supply of the radiometal in a biocompatible carrier, dissolution occurs to give the desired radiopharmaceutical composition.

Preferred aspects of the chelator conjugate in the fifth aspect are as described in the second aspect of the present invention (above).

By the term “kit” is meant one or more non-radioactive pharmaceutical grade containers, comprising the necessary chemicals to prepare the desired radiopharmaceutical composition, together with operating instructions. The kit is designed to be reconstituted with the desired radiometal to give a solution suitable for human administration with the minimum of manipulation.

The sterile, solid form is preferably a lyophilised solid.

For ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container (eg. a septum-sealed vial) containing the chelator conjugate in either free base or acid salt form, together with a biocompatible reductant such as sodium dithionite, sodium bisulfite, ascorbic acid, formamidine sulfinic acid, stannous ion, Fe(II) or Cu(I). The biocompatible reductant is preferably a stannous salt such as stannous chloride or stannous tartrate. Alternatively, the kit may optionally contain a non-radioactive metal complex which, upon addition of the technetium, undergoes transmetallation (i.e. metal exchange) giving the desired product. The non-radioactive kits may optionally further comprise additional components such as a transchelator, radioprotectant, antimicrobial preservative, pH-adjusting agent or filler—as defined above.

In a sixth aspect, the present invention provides a method of imaging the human or animal body which comprises generating an image of at least a part of said body to which the imaging agent of the first aspect, or the composition of the fourth aspect has distributed using PET or SPECT, wherein said imaging agent or composition has been previously administered to said body.

Preferred aspects of the imaging agent or composition in the sixth aspect are as described in the first and fourth aspects respectively of the present invention (above). The method of the sixth aspect is preferably carried out where the part of the body is disease state where abnormal apoptosis is involved. By the term “abnormal apoptosis” is meant dysregulation of the programmed cell death (PCD) process. Such dysregulation has been implicated in a number of disease states, including those associated with the inhibition of apoptosis, such as cancer and autoimmune disorders, and those associated with hyperactive apoptosis, including: neurodegenerative diseases; haematologic diseases; AIDS; ischaemia; allograft rejection and cardiology (myocardial infarction, atherosclerosis and/or cardiotoxicity follow drug therapy). The visualization and quantitation of apoptosis is therefore useful in the diagnosis of such apoptosis-related pathophysiology.

The imaging method of the sixth aspect may optionally be carried out repeatedly to monitor the effect of treatment of a human or animal body with a drug, said imaging being effected before and after treatment with said drug, and optionally also during treatment with said drug. Therapeutic treatments for these diseases aim to restore balanced apoptosis, either by stimulating or inhibiting the PCD process as appropriate. Of particular interest is early monitoring of the efficacy of cancer therapy to ensure that malignant growth is controlled before the condition becomes terminal.

In a seventh aspect, the present invention provides the use of the imaging agent of the first aspect, the composition of the fourth aspect, or the kit of the fifth aspect in a method of diagnosis of the human or animal body.

In an eighth aspect, the present invention provides a method of diagnosis of the human or animal body which comprises the method of imaging of the sixth aspect.

Preferred aspects of the imaging agent or composition in the seventh and eighth aspects are as described in the first and fourth aspects respectively of the present invention (above). The diagnosis of the human or animal body of both aspects is preferably of a disease state where abnormal apoptosis is involved. Such “abnormal apoptosis” is as described in the sixth aspect (above).

The invention is illustrated by the non-limiting Examples detailed below. Examples 1 to 3 provide the synthesis of Chelator 1 (a diaminedioxime) of the invention, and Example 4 the synthesis of Chelator 1A (a diaminedioxime functionalised with glutaric acid) and synthesis of the corresponding active ester Chelator 1A-TFTP ester. Example 5 the synthesis of Chelator 1B (a diaminedioxime functionalised with glutaryl-amino-PEG12 propionic acid). Example 6 provides the synthesis of a Boc-protected tetra-amine chelator of the invention (Chelator 2A). Example 7 provides the synthesis of a HYNIC-duramycin conjugate (prior art) for comparative purposes. Example 8 provides the synthesis of duramycin functionalised with Chelator 1A (Conjugate 3A and Conjugate 3B). Example 9 provides the synthesis of cinnamycin with Chelator 1A (Conjugate 5). Example 10 provides the synthesis of duramycin with Chelator 1B (Conjugate 6). Example 11 provides the synthesis of cinnamycin with Chelator 1B (Conjugate 6). Example 12 provides the synthesis of duramycin functionalised with Chelator 2A (Conjugate 2A and Conjugate 2B) and Example 13 of cinnamycin with Chelator 2A (Conjugate 4). Example 14 provides the radiolabelling of the chelator conjugates of the invention with the radiometal ^{99m}Tc. The ^{99m}Tc complexes form as a single species with high RCP. That is an advantage over HYNIC, where multiple species form when HYNIC/phosphine/tricine labelling is used. The procedure is simple, with efficient labeling at room temperature. The RCP is very good, even at high radioactive concentration (>90% RCP at >500 MBq/mL).

Example 15 provides determination of the site of conjugation of a chelator and Example 16 demonstrates that the site of conjugation of a chelator has a significant effect on the binding affinity for phosphatidylethanolamine, with a factor of 18 difference (K_{d} 5 nM vs 90 nM). This provides evidence that attachment of the radiometal complex at the N-terminus (Cys^{a} of Formula II) is preferred over attachment at Xaa of Formula II. The EL4 lymphoma mouse xenograft tumour model of Example 17 has been used as a model to mimic the apoptotic response following chemotherapy. Therapy-treated mice (etoposide/cyclophosphamide) showed a 4 fold increase in tumour apoptosis compared to vehicle control treated animals. The biodistribution results of Example 17 show a higher uptake of each agent in chemotherapy-treated tumours, while correlation analysis suggests a trend of higher binder uptake in tumours with higher levels of apoptosis. ^{99m}Tc-[Conjugate 5] had similar tumour and improved liver performance vs ^{99m}Tc-[Conjugate 3A]. ^{99m}Tc-[Conjugate 2A] shows similar tumour but inferior lung performance vs ^{99m}Tc-[Conjugate 5]. Repeat imaging studies with ^{99m}Tc-[Conjugate 5] showed a consistent increase in tumour: muscle ratios following therapy. Example 18 shows that a PEG linker group is advantageous in reducing liver background and increasing urinary excretion in vivo.

### Abbreviations.

Conventional single letter or 3-letter amino acid abbreviations are used.

% id: Percentage injected dose

### Ac: Acetyl.

Acm: Acetamido methyl.

### ACN: Acetonitrile.

Boc: tent-Butyloxycarbonyl.

### Bz: Benzyl.

### DCM: Dichloromethane.

### DIPEA: N-Diisopropylethylamine.

### DMF: N,N-Dimethylformamide.

### DMSO: Dimethylsulfoxide.

### Fmoc: 9-Fluorenylmethoxycarbonyl.

Glut: Glutaric acid.
HATU: O-(7-Azabenzotriazol-1-yl)-N,N,N′,N′-tetramethyluronium hexafluorophosphate.
HOAt: 7-Aza-1-hydroxybenzotriazole.
HPLC: High performance liquid chromatography.
IBX: 1-Hydroxy-1,2-benziodoxole-3(1H)-one-1-oxide.
MDP: Methylenediphosphonic acid.
NaPABA: Sodium para-aminobenzoate.

### NHS: N-Hydroxy-succinimide.

### NMM: N-Methylmorpholine.

NMP: 1-Methyl-2-pyrrolidinone.
PBS: Phosphate-buffered saline.

### PEG12: —(OCH₂CH₂)₁₂—.

PyAOP: (7-Azabenzotriazole-1-yl-oxy-tris-pyrrolidino-phosphonium hexafluorophosphate.
PyBOP: Benzotriazol-1-yl-oxytripyrrolidinophosphonium hexafluorophosphate.
RAC: Radioactive concentration.
RCP: Radiochemical purity.
tBu: tert-Butyl.
TFA: Trifluoroacetic acid.

### TFTP: Tetrafluorothiophenol.

### THF: Tetrahydrofuran.

### TIS: Triisopropylsilane.

### Trt: Trityl.

TABLE 1
Compounds of the Invention.
Formula II (with bridges as specified in the first aspect):
Cys^{a}-Xaa-Gln-Ser^{b}-Cys^{c}-Ser^{d}-Phe-Gly-Pro-Phe-Thr^{c}-Phe-
Val-Cys^{b}-(HO-Asp)-Gly-Asn-Thr^{a}-Lys^{d}

| Name | Structure |
|---|---|
| LBP1 | Duramycin Formula II, where Xaa = Lys. |
| LBP2 | Cinnamycin Formula II, where Xaa = Arg. |
| Conju- | [HYNIC]-LBP1, with HYNIC attached at either of Cys^{a} |
| gate 1 | & Xaa. (A mixture of mono- and bis- fuctionalised species). |
| (prior art) | |
| Conju- | [HYNIC]-LBP1, with HYNIC attached at Cys^{a} |
| gate 1A | (mono-functionalised species). |
| Conju- | [HYNIC]-LBP1, with HYNIC attached at Xaa |
| gate 1B | (mono-functionalised species). |
| Conju- | [Chelator 2A]-LBP1, with Chelator 2A attached at Cys^{a} |
| gate 2A | (mono-functionalised species). |
| Conju- | [Chelator 2A]-LBP1, with Chelator 2A attached at Xaa |
| gate 2B | (mono-functionalised species). |
| Conju- | [Chelator 1A]-LBP1, with Chelator 1A attached at either Cys^{a} |
| gate 3A | and Xaa (mixture of mono-functionalised species). |
| Conju- | [Chelator 1A]-LBP1, with Chelator 1A attached at both Cys^{a} |
| gate 3B | and Xaa bis-functionalised species). |
| Conju- | [Chelator 2A]-LBP2, with Chelator 2A attached at Cys^{a}. |
| gate 4 | (mono-functionalised species). |
| Conju- | [Chelator 1A]-LBP2, with Chelator 1A attached at Cys^{a}. |
| gate 5 | (mono-functionalised species). |
| Conju- | [Chelator 1B]-LBP1, with Chelator 1B attached at either Cys^{a} |
| gate 6 | and Xaa (mixture of mono-functionalised species). |
| Conju- | [Chelator 1B]-LBP2, with Chelator 1B attached at Cys^{a} |
| gate 7 | (mono-functionalised species). |

### Example 1

### Synthesis of 1,1,1-tris(2-aminoethyl)methane

### Step 1(a): 3(methoxycarbonylmethylene)glutaric acid dimethylester

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether 1:1, 600 ml. Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3-(methoxycarbonylmethylene)glutaric acid dimethylester (89.08 g, 53%).

NMR ¹H(CDCl₃): δ 3.31 (2H, s, CH₂), 3.7 (9H, s, 3×OCH₃), 3.87 (2H, s, CH₂), 5.79 (1H, s, ═CH,) ppm.

NMR ¹³C(CDCl₃), δ 36.56, CH₃, 48.7, 2×CH₃, 52.09 and 52.5 (2×CH₂); 122.3 and 146.16 C═CH; 165.9, 170.0 and 170.5 3×COO ppm.

### Step 1(b): Hydrogenation of 3-(methoxycarbonylmethylene)glutaric acid dimethylester

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H, s, 3×CH₃).

NMR ¹³C(CDCl₃), [symbol] 28.6, CH; 37.50, 3×CH₃; 51.6, 3×CH₂; 172.28, 3×COO.

### Step 1(c): Reduction and Esterification of Trimethyl Ester to the Triacetate

Under an atmosphere of nitrogen in a 3 necked 2 L round bottomed flask lithium aluminium hydride (20 g, 588 mmol) in THF (400 ml) was treated cautiously with tris(methyloxycarbonylmethyl)methane (40 g, 212 mmol) in THF (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the THF. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulfate, and concentrated in vacuo to afford an oil. The oil was Kugelrohr distilled in high vacuum to give tris(2-acetoxyethyl)methane (45.3 g, 95.9%) as an oil. Bp. 220° C. at 0.1 mmHg.

NMR ¹H(CDCl₃), δ 1.66 (7H, m, 3×CH₂, CH), 2.08 (1H, s, 3×CH₃); 4.1 (6H, t, 3×CH₂O).

NMR ¹³C(CDCl₃), Δ 20.9, CH₃; 29.34, CH; 32.17, CH₂; 62.15, CH₂O; 171, CO.

### Step 1(d): Removal of Acetate Groups from the Triacetate

Tris(2-acetoxyethyl)methane (45.3 g, 165 mM) in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide by 170-180 0.2 mm. The bulbs containing the acetamide were washed clean and the distillation continued. Tris(2-hydroxyethyl)methane (22.53 g, 92%) distilled at by 220° C. 0.2 mm.

NMR ¹H(CDCl₃), δ 1.45 (6H, q, 3×CH₂), 2.2 (1H, quintet, CH); 3.7 (6H, t 3×CH₂OH); 5.5 (3H, brs, 3×OH).

NMR ¹³C(CDCl₃), Δ 22.13, CH; 33.95, 3×CH₂; 57.8, 3×CH₂OH.

### Step 1(e): Conversion of the triol to the tris(methanesulphonate)

To an stirred ice-cooled solution of tris(2-hydroxyethyl)methane (10 g, 0.0676 mol) in dichloromethane (50 ml) was slowly dripped a solution of methanesulfonyl chloride (40 g, 0.349 mol) in dichloromethane (50 ml) under nitrogen at such a rate that the temperature did not rise above 15° C. Pyridine (21.4 g, 0.27 mol, 4 eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulfate, filtered and concentrated in vacuo to give tris[2-(methylsulphonyloxy)ethyl]methane contaminated with excess methanesulfonyl chloride. The theoretical yield was 25.8 g.

NMR ¹H(CDCl₃), δ 4.3 (6H, t, 2×CH₂), 3.0 (9H, s, 3×CH₃), 2 (1H, hextet, CH), 1.85 (6H, q, 3×CH₂).

### Step 1(f): Preparation of 1,1,1-tris(2-azidoethyl)methane

A stirred solution of tris[2-(methylsulfonyloxy)ethyl]methane [from Step 1(e), contaminated with excess methylsulfonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulfate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution. The ethanol solution of triazide was used directly in Step 1(g).

### CARE: DO NOT REMOVE ALL THE SOLVENT AS THE AZIDE IS POTENTIALLY EXPLOSIVE AND SHOULD BE KEPT IN DILUTE SOLUTION AT ALL TIMES.

Less than 0.2 ml of the solution was evaporated in vacuum to remove the ethanol and an NMR run on this small sample:

NMR ¹H(CDCl₃), δ 3.35 (6H, t, 3×CH₂), 1.8 (1H, septet, CH,), 1.6 (6H, q, ³×CH₂).

### Step 1(g): Preparation of 1,1,1-tris(2-aminoethyl)methane

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine.

Caution: Unreduced Azide could Explode on Distillation.

The reaction was filtered through a celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 82.7% overall yield from the triol).

NMR ¹H(CDCl₃), δ 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂.), 31.0 (CH).

### Example 2

### Preparation of 3-chloro-3-methyl-2-nitrosobutane

A mixture of 2-methylbut-2-ene (147 ml, 1.4 mol) and isoamyl nitrite (156 ml, 1.16 mol) was cooled to −30° C. in a bath of cardice and methanol and vigorously stirred with an overhead air stirrer and treated dropwise with concentrated hydrochloric acid (140 ml, 1.68 mol) at such a rate that the temperature was maintained below −20° C. This requires about 1 h as there is a significant exotherm and care must be taken to prevent overheating. Ethanol (100 ml) was added to reduce the viscosity of the slurry that had formed at the end of the addition and the reaction stirred at −20 to −10° C. for a further 2 h to complete the reaction. The precipitate was collected by filtration under vacuum and washed with 4×30 ml of cold (−20° C.) ethanol and 100 ml of ice cold water, and dried in vacuo to give 3-chloro-3-methyl-2-nitrosobutane as a white solid. The ethanol filtrate and washings were combined and diluted with water (200 ml) and cooled and allowed to stand for 1 h at −10° C. when a further crop of 3-chloro-3-methyl-2-nitrosobutane crystallised out. The precipitate was collected by filtration and washed with the minimum of water and dried in vacuo to give a total yield of 3-chloro-3-methyl-2-nitrosobutane (115 g 0.85 mol, 73%)>98% pure by NMR.

NMR ¹H(CDCl₃), As a mixture of isomers (isomer1, 90%) 1.5 d, (2H, CH₃), 1.65 d, (4H, 2×CH₃), 5.85, q, and 5.95, q, together 1H. (isomer2, 10%), 1.76 s, (6H, 2×CH₃), 2.07 (3H, CH₃).

### Example 3

### Synthesis of bis[N-(1,1-dimethyl-2-N-hydroxyimine propyl)-2-aminoethyl]-(2-aminoethyl)methane (Chelator 1)

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24 (6H, s, 2×CH₃), 1.3 (6H, s, 2×CH₃), 1.25-1.75 (7H, m, 3×CH₂CH), (3H, s, 2×CH₂), 2.58 (4H, m, CH₂N), 2.88 (2H, t CH₂N₂), 5.0 (6H, s, NH₂, 2×NH, 2×OH).

NMR ¹H((CD₃)₂SO) δ1.14×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N; 160.3; C═N.

HPLC conditions: flow rate 8 ml/min using a 25 mm PRP column [A=3% ammonia solution (sp.gr=0.88)/water; B=Acetonitrile].

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### Example 4

### Synthesis of Tetrafluorothiophenyl ester of Chelator 1-glutaric acid (Chelator 1A-TFTP Ester)

### a) Synthesis of [Chelator 1]-glutaric acid (Chelator 1A)

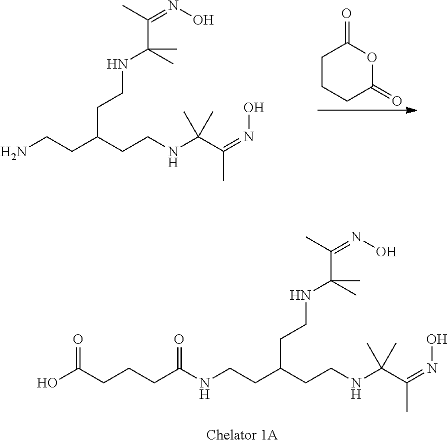 [SMILES: C/C(=N/O)C(C)(C)NCCC(CCN)CCNC(C)(C)/C(C)=N\O ; O=C1CCCC(=O)O1 ; C/C(=N/O)C(C)(C)NCCC(CCNC(=O)CCCC(=O)O)CCNC(C)(C)/C(C)=N\O]

Chelator 1 (100 mg, 0.29 mmol) was dissolved in DMF (10 ml) and glutaric anhydride (33 mg, 0.29 mmol) added by portions with stirring. The reaction was stirred for 23 hours to afford complete conversion to the desired product. The pure acid was obtained following RP-HPLC in good yield.

### b) Synthesis of Chelator 1A-TFTP Ester

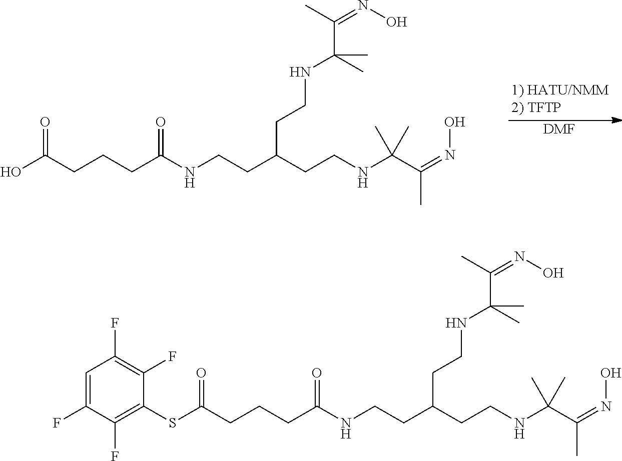

To Chelator 1A (from Step a; 300 mg, 0.66 mmol) in DMF (2 ml) was added HATU (249 mg, 0.66 mmol) and NMM (132 μL, 1.32 mmol). The mixture was stirred for 5 minutes then tetrafluorothiophenol (0.66 mmol, 119 mg) was added. The solution was stirred for 10 minutes then the reaction mixture was diluted with 20% acetonitrile/water (8 ml) and the product purified by RP-HPLC yielding 110 mg of the desired product following freeze-drying.

### Example 5

### Synthesis of Chelator 1-glutaryl-amino-PEG12 propionic acid (Chelator 1B)

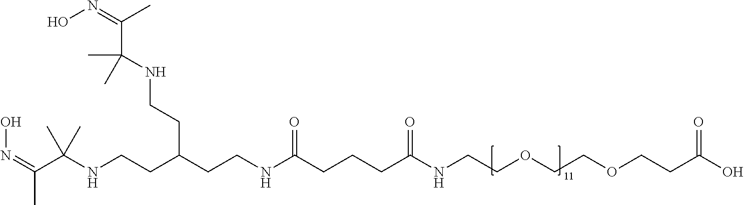

Boc-amino-PEG12 propionic acid (Polypure; 45 mg, 0.060 mmol) was treated with TFA/water (19:1) (1 mL) for 30 min. The TFA was then evaporated in vacuo and the residue dried in vacuo overnight affording 52 mg crude amino-PEG12 propionic acid. Chelator 1A (46 mg, 0.10 mmol) and PyAOP (31 mg, 0.060 mmol) were dissolved in NMP (1 mL). DIPEA (42 μL, 0.24 mmol) was added and the solution shaken for 5 min and added to amino-PEG12 propionic acid (0.06 mmol). The reaction mixture was shaken overnight. Additional Chelator 1A (0.03 mmol) was added to the reaction mixture and after 1 h the mixture was diluted with water/0.1% TFA (7 mL) and the product purified by preparative RP-HPLC.

### Purification and Characterization

Purification by RP-HPLC (gradient: 10-30% B over 40 min, t_{R}: 34.5 min) afforded 44 mg (67% yield) of Chelator 1B after lyophilisation. Chelator 1B was characterized by LC-MS (gradient: 10-40% B over 5 min, t_{R}: 2.2 min; calcd. m/z 1057.7 [MH]⁺. found m/z 1058.0).

### Example 6

### Synthesis of Chelator 2A

Step (a): Diethyl [2-(benzyloxy)ethyl]malonate

The compound was prepared by a modification of the method of Ramalingam et al Tetrahedron, 51, 2875-2894 (1995)]. Thus, sodium (1.20 g) was dissolved in absolute ethanol (25 ml) under argon. Diethyl malonate (14.00 g) was added and the mixture was refluxed for 30 min. Benzyl bromoethyl ether (10 g) was added and the mixture was stirred at reflux for 16 hours. The ethanol was removed by rotary evaporation and the residue was partitioned between ether (100 ml) and water (50 ml). The ethereal layer was washed with water (3×50 ml) and dried over sodium sulfate. The ether was removed by rotary evaporation and the residue was distilled in vacuo. The fraction distilling at 40-55° C. was discarded (unreacted diethyl malonate). The product distilled at 140-150° C. (1 mm), [lit. by 138-140 C (1 mm)]. The yield was 12.60 g of colourless oil.

¹H NMR (270 MHz, CDCl₃, 25° C., TMS) δ=7.28 (m, 5HC₆H₅), 4.47 (s, 2H, CH₂-Ph), 4.16 (m, 4H, COOCH₂), 3.58 (t, 1H, CH), 3.50 (t, 2H, O—CH₂—CH₂), 2.21 (t, 2H, O—CH₂—CH₂), 1.20 (t, 6H, COOCH₂—CH₃). ¹³C NMR (67.5 MHz, CDCl₃, 25° C., TMS) δ=169.20 (CO), 138.10, 128.60, 127.80 (aromatic), 73.00 (CH₂Ph), 67.30 (O—CH₂—CH₂), 61.70 (COOCH₂), 49.10 (CH), 28.90 (O—CH₂—CH₂), 14.10 (COOCH₂CH₃).

### Step (b): N N′-Bis(2-aminoethyl)-2-(2-benzyloxy-ethyl)malonamide

Diethyl [2-(benzyloxy)ethyl]malonate (4.00 g) was added to ethylene diamine (30 ml) and the solution was stirred at room temperature for two days. The excess ethylene diamine was removed by rotary evaporation and the residue was dried under high vacuum for 2 days to give a yellow oil (4.28 g) that crystallized on standing. The product still contained traces of ethylenediamine, as detected in the NMR spectra.

¹H NMR (270 MHz, CDCl₃, 25° C., TMS) δ=7.74 (br t, 2H, CO—NH), 7.32 (m, 5H, C₆H₅), 4.46 (s, 2H, CH₂-Ph), 3.50 (t, 2H, OCH₂—CH₂—), 3.33 (t 1H, CH), 3.23 (m, 4H, CO—NH—CH₂), 2.74 (t, 4H, CH₂—NH₂) 2.18 (q, 2H, O—CH₂—CH₂—) 1.55 (br s 4H, NH₂).

¹³C NMR (67.5 MHz, CDCl₃, 25° C., TMS) δ=171.10 (CO), 138.20, 128.30, 127.70 (aromatic), 73.00 (CH₂-Ph), 67.80 (O—CH₂—CH₂), 51.40 (CH), 42.40 (CO—NH—CH₂), 41.20 (CH₂—NH₂), 31.90 (O—CH₂—CH₂—).

### Step (c): N,N′-Bis(2-amino-ethyl)-2-(2-benzyloxyethyl)-1,3-diaminopropane

N,N′-Bis-(2-aminoethyl)-2-(2-benzyloxy-ethyl)malonamide (3.80 g) was dissolved in THF (20 ml) and the flask was immersed in an ice bath. The flask was flushed with argon and THF borane complex (80 ml, 1M in THF) was added through a syringe. The reaction mixture was allowed to warm up to room temp. and then stirred at 40° C. for 2 days and refluxed for 1 h. Methanol (50 ml) was added dropwise and the solution was stirred at 40° C. overnight. The solvents were removed by rotary evaporator and the residue was dissolved in methanol (20 ml). Sodium hydroxide (10 g in 15 ml of water) was added and the methanol was boiled away. A colourless oil separated that was extracted into CH₂Cl₂ (3×50 ml). The solution was dried over Na₂SO₄. Removal of the solvent gave 3.40 g of colourless oil.

¹H NMR (270 MHz, CDCl₃, 25° C., TMS) δ=7.34 (m, 5H, C₆H₅), 4.49 (s, 2H, CH₂-Ph), 3.55 (t, 2H, OCH₂—CH₂—), 2.76 (t, 4H, N—CH₂), 2.63 (m, 8H, N—CH₂), 1.84 (m, 1H, CH), 1.58 (m, 2H, CH—CH₂—CH₂—O), 1.41 (br s, 6H, NH). ¹³C NMR (67.5 MHz, CDCl₃, 25° C., TMS) δ=138.60, 128.30, 127.60 (aromatic), 72.80 (CH₂-Ph), 68.70 (O—CH₂—CH₂), 53.50 (N—CH₂), 52.80 (N—CH₂), 41.60 (N—CH₂) 36.40 (CH), 31.30 (CH—CH₂—CH₂—O). MS-EI: 295 [M+H]⁺, (calcd.: 295.2).

### Step (d): N,N′-Bis 2-tert-butoxycarbonylamino-ethyl)-2-(2-benzyloxyethyl)-1,3-di(tert-butoxycarbonylamino)propane

N,N′-Bis(2-aminoethyl)-2-(2-benzyloxy-ethyl)-1,3-diaminopropane (3.30 g) was dissolved in CH₂Cl₂ (100 ml) and triethylamine (5.40 g) and tert-butyl dicarbonate (10.30 g) were added. The reaction mixture was stirred at room temp. for 2 days. The mixture was washed with water (100 ml), citric acid solution (100 ml, 10% in water) and with water (2×100 ml). The organic layer was dried over Na₂SO₄, and the solvent was removed by rotary evaporation giving a yellow oil which was dried to a constant mass under high vacuum. The crude product (7.70 g) was purified on a silica gel column (250 g, 230-400 mesh, CH₂Cl₂, CH₂Cl₂-Et₂O 1:1) to give 6.10 g (78.3%) of a clear oil.

¹H NMR (270 MHz, CDCl₃, 25° C., TMS) δ=7.32 (m, 5H, C₆H₅), 5.12 (br d, 2H, NH), 4.47 (s, 2H, CH₂-Ph), 3.49 (t, 2H, OCH₂—CH₂—), 3.24 (br, 12H, N—CH₂), 2.14 (br, 1H, CH), 1.59 (m, 2H, CH—CH₂—CH₂—O) 1.45 (s, 18H, t-Bu), 1.42 (s, 18H, t-Bu). ¹³C NMR (67.5 MHz, CDCl₃, 25° C., TMS) δ=155.90 (NH—CO), 138.20, 128.30 127.60, 127.50 (aromatic), 79.90, 78.90 (CMe₃), 72.80 (CH₂-Ph), 68.00 (O—CH₂—CH₂), 50.00 (br, N—CH₂), 46.90 (br, N—CH₂), 39.20 (N—CH₂), 34.40 (br, CH), 29.80 (CH—CH₂—CH₂—O), 28.30 (t-Bu). MS-EI: 695 [M+H]⁺, (calcd.: 695.5)

### Step (e): N,N′-Bis(2-tert-butoxycarbonylamino-ethyl)-2-(2-hydroxyethyl)-1,3-di(tert-butoxycarbonylamino)propane

N,N′-Bis(2-tert-butoxycarbonylamino-ethyl)-2-(2-benzyloxy-ethyl)-1,3-di(tert-butoxycarbonylamino)propane (3.16 g) was dissolved in absolute ethanol (100 ml) and Pd on activated carbon (1.00 g, dry, 10%) was added. The mixture was hydrogenated in a Parr hydrogenation apparatus at 35 psi for two days. The catalyst was filtered off, washed with ethanol (3×20 ml). The ethanol was removed by rotary evaporation to give a colourless oil that was dried to a constant mass (2.67 g, 97.1%) under high vacuum.

¹H NMR (270 MHz, CDCl₃, 25° C., TMS) δ=5.25 (br d, 2H, NH), 3.69 (t, 2H, OCH₂—CH₂—), 3.28 (br, 12H, N—CH₂), 2.71 (br, OH), 2.23 (br, 1H, CH), 1.56 (shoulder, m, 2H, CH—CH₂—CH₂—O) 1.48 (s, 18H, t-Bu), 1.44 (s, 18H, t-Bu). ¹³C NMR (67.5 MHz, CDCl₃, 25° C., TMS) δ=156.10 (NHCO), 80.00, 79.20 (CMe₃), 59.60 (O—CH₂—CH₂), 49.90 (br, N—CH₂), 47.00 (br, N—CH₂), 39.34 (N—CH₂), 33.80 (CH), 32.30 (CH—CH₂—CH₂—O), 28.30 (t-Bu). MS-EI: 605 [M+H]⁺, (calcd.: 605.4).

### Step (f): N,N′-Bis(2-tert-butoxycarbonylamino-ethyl)-2-(2-carboxymethyl)-1,3-di(tert-butoxycarbonylamino)propane (Boc-protected Chelator 2A)

The method of Mazitschek et al [Ang. Chem. Int. Ed., 41, 4059-4061 (2002)] was used. Thus, N,N′-Bis(2-tert-butoxycarbonylamino-ethyl)-2-(2-hydroxyethyl)-1,3-di(tert-butoxycarbonylamino)propane (2.60 g) was dissolved in DMSO (15 ml) and 1-hydroxy-1,2-benziodoxole-3(1H)-one-1-oxide (IBX, 3.50 g) was added. The mixture was stirred at room temp. for 1 hour then N-hydroxysuccinimide (2.50 g) was added. The reaction mixture was stirred at room temp. for 2 days. Sodium hydroxide solution (2M, 40 ml) was added and the mixture was stirred at room temp. for 4 hours. The solution was immersed in an ice bath and was acidified with 2M hydrochloric acid to pH 2. The aqueous layer was extracted with ether (4×100 ml) and the combined ether extracts were washed with water (3×50 ml). The ethereal layer was dried over Na₂SO₄ and the solvent was removed by rotary evaporation to give a yellow solid residue that contained the product and 2-iodosobenzoic acid. Most of the iodosobenzoic acid (2.1 g) was removed by crystallization from chloroform-hexanes (1:3) (80 ml). Evaporation of the chloroform-hexanes mother liquor gave a yellow oil (3 g) that was loaded on a silica column (300 g, CH₂Cl₂-Et₂₀, 1:1). The remaining iodosobenzoic acid was eluted with ether. The product was eluted with ether-methanol (9:1). The fractions containing the product were combined and removal of the solvent gave 1.5 g of pale yellow oil. This was re-chromatographed on a silica column (50 g, Et₂O). The product was eluted with ether-acetic acid (95:5). The fractions containing the product were combined and the solvent was removed by rotary evaporation to give an oil that was dried under high vacuum. The yield was 1.10 g (41.3%).

¹H NMR (270 MHz, CDCl₃, 25° C., TMS) δ=7.61 (br s, 1H, COOH), 5.19 (br d, 2H, NH), 3.22 (br, 12H, N—CH₂), 2.47 (br m, 1H, CH), 2.26 (br, 2H, CH—CH₂—COOH), 1.41 (s, 18H, t-Bu), 1.37 (s, 18H, t-Bu). ¹³C NMR (67.5 MHz, CDCl₃, 25° C., TMS) δ=175.90 (COOH), 156.10 (NHCO), 80.40, 79.10 (CMe₃), 49.50 (N—CH₂), 46.80 (N—CH₂), 39.00 (N—CH₂), 34.70 (CH—CH₂—COOH), 34.20 (CH—CH₂—COOH), 28.30, 28.20 (t-Bu). MS-EI: 619 [M+H]⁺, (calcd.: 619.4).

### Example 7

### Synthesis of HYNIC-Duramycin (Conjugate 1A and Conjugate 1B)

Duramycin (Sigma-Aldrich; 5.0 mg, 2.5 μmol) and N-Boc-HYNIC succinimidyl ester (ABX Advanced Biochemical Compounds; 1.0 mg, 2.8 μmol) were dissolved in DMF (1 ml) and DIPEA (2.0 μL, 13 μmol) was added to the mixture. The reaction progress was monitored by LC-MS analysis. Addition of HOAt (1.1 eq) after 3 hrs, in order to drive the sluggish reaction, afforded ˜60% product formation overnight. Additional N-Boc-HYNIC succinimidyl ester (1 eq) and HOAt (2 eq) were needed to obtain ˜80% HYNIC-conjugate formation after one subsequent day at room temperature and 3 days at 4° C. Two baseline separated peaks corresponding to mono-conjugates were observed by LC-MS analysis in addition to the bis-conjugate (−35%).

### Purification and Characterisation.

Water/0.1% TFA (4 ml) was added to the reaction mixture and the two mono-conjugated products were purified by preparative RP-HPLC (gradient: 0% B over 15 min; 0-45% B over 10 min; 45-60% B over 40 min, t_{R}: 47.9 and 49.3 min) and then Boc-deprotected in TFA affording two mono-conjugated Duramycin isomers, in 1.7 mg and 1.1 mg yield, respectively.

The two isomers were characterised by LC-MS (gradient: 20-40% B over 5 min, t_{R}: 2.8 min (Conjugate 1A). found m/z: 1074.8, expected MH₂²⁺: 1074.4, t_{R}: 2.9 min (Conjugate 1B). found m/z: 1074.8, expected MH₂²⁺: 1074.4.

### Example 8

### Synthesis of [Chelator 1A]-Duramycin Mono-conjugate (Conjugate 3A) and [Chelator 1A]-Duramycin bis-Conjugate (Conjugate 3B)

Chelator 1A (Example 4; 3.0 mg, 6.6 μmol), PyBOP (2.6 mg, 5.0 μmol) and DIPEA (1.7 μL, 9.7 μmol) were dissolved in NMP (0.7 ml). The mixture was shaken for 5 min and added to a solution of Duramycin (Sigma-Aldrich; 5.0 mg, 2.5 μmol) in NMP (0.5 ml). The reaction mixture was shaken for 40 min, and then diluted with water/0.1% TFA (6 ml) and the product purified using preparative HPLC.

Purification by preparative HPLC (gradient: 5-35% B over 40 min where A=H₂O/0.1% HCOOH and B=ACN/0.1% HCOOH) afforded 2.5 mg pure Conjugate 3A (yield 41%) and 1.7 mg pure Conjugate 3B (yield 28%).

The purified Conjugate 3A was analysed by analytical LC-MS (gradient: 25-35% B over 5 min, t_{R}: 1.93 min. found m/z: 1227.0, expected MH₂²⁺: 1226.6).

The purified Conjugate 3B was analysed by analytical LC-MS (gradient: 25-35% B over 5 min, t_{R}: 2.35 min. found m/z: 1446.7, expected MH₂²⁺: 1446.3).

Separation of the two mono-conjugates (Conjugate 3A) could not be achieved using either analytical or preparative HPLC. In each case the two regioisomers eluted as one single peak.

### Example 9

### Synthesis of [Chelator 1A]-Cinnamycin Conjugate (Conjugate 5)

Cinnamycin (Sigma-Aldrich; 2.0 mg, 1.0 μmol), Chelator 1A (Example 4; 0.9 mg, 1.5 μmol) and DIPEA (0.5 μL, 2.9 μmol) were dissolved in a solution of NMP (0.2 ml), DMF (0.2 ml) and DMSO (0.6 ml). The reaction mixture was shaken overnight. The mixture was then diluted with 10% ACN/water/0.1% TFA (7 ml) and the product purified using preparative HPLC.

### Purification and Characterisation

Purification by preparative HPLC (gradient: 20-40% B over 40 min) afforded 1.9 mg pure Conjugate 5 (yield 78%). The purified material was analysed by analytical LC-MS (gradient: 20-40% B over 5 min, t_{R}: 2.86 min. found m/z: 1241.0, expected MH₂²⁺: 1240.6).

### Example 10

### Synthesis of [Chelator 1B]-Duramycin Conjugate (Conjugate 6)

Chelator 1B (Example 5; 1.6 mg, 1.5 μmol), PyBOP (0.4 mg, 0.8 μmol) and DIPEA (1 μL, 6 μmol) were dissolved in NMP (0.5 mL). The mixture was shaken for 5 min and added to a solution of duramycin (3.0 mg, 1.5 μmol) in NMP (0.5 mL). The reaction mixture was shaken for 30 min. Two additional aliquots of activated Chelator 1B (2×1.6 mg) were added at 30 min intervals. The mixture was diluted with water/0.1% TFA (6 mL) and the product purified using preparative RP-HPLC.

### Purification and Characterisation

Purification by preparative HPLC (gradient: 20-50% B over 40 min where A=water/0.1% ammonium acetate and B=ACN) afforded 3.9 mg pure Conjugate 6 (yield 87%). The purified material was analysed by LC-MS (gradient: 20-40% B over 5 min, t_{R}: 2.89 min. found m/z: 1526.5, expected MH₂²⁺: 1526.2).

### Example 11

### Synthesis of [Chelator 1B]-Cinnamycin Conjugate (Conjugate 7)

Chelator 1B (Example 5; 4.8 mg, 4.4 μmol), PyBOP (2.1 mg, 4.0 μmol) and DIPEA (2.3 μL, 13.2 μmol) were dissolved in DMF (0.5 mL). The mixture was shaken for 5 min and added to a solid cinnamycin (4.5 mg, 2.2 μmol). Additional pre-activated Chelator 1B was added after 2 h and after 3.5 h in order to drive the reaction close to completion within 4 h. The mixture was diluted with 20% ACN/water/0.1% TFA (8 mL) and the product purified using preparative RP-HPLC.

### Purification and Characterization

Purification by preparative RP-HPLC (gradient: 25-35% B over 40 min; t_{R} 38.6 min) afforded 3.9 mg purified Conjugate 7 (yield 58%).

The purified material was analysed by LC-MS (gradient: 20-40% B over 5 min: t_{R} 2.9 min. found m/z: 1028.0, expected MH₂²⁺: 1027.5 (purity ˜93.5%, ˜3% unreacted starting material).

### Example 12

### Synthesis of [Chelator 2A]-Duramycin Conjugate (Conjugate 2A and Conjugate 2B)

Duramycin (Sigma-Aldrich; 7.5 mg, 3.8 μmol), Boc-protected Chelator 2A (Example 6; 5.0 mg, 6.9 μmol), HOAt (1.9 mg, 8.8 μmol) and DIPEA (4.1 μL, 20.0 μmol) were dissolved in NMP (1.5 ml). The reaction mixture was shaken overnight. The mixture was then diluted with 20% ACN/water/0.1% TFA (6 ml) and the product purified using preparative HPLC.

### Purification and Characterisation.

Purification by preparative HPLC (gradient: 0% B over 10 min; 0-30% B over 5 min; 30-70% B over 40 min, t_{R}: 42.4 and 45.0 min), followed by Boc-deprotection in TFA afforded two mono-conjugated Duramycin isomers, in 2.0 mg and 0.4 mg yield, respectively.

The two isomers were characterized by LC-MS (gradient: 20-60% B over 5 min, t_{R}: 1.7 min (Conjugate 2A). found m/z: 1107.5, expected MH₂²⁺: 1107.0, t_{R}: 1.6 min (Conjugate 2B). found m/z: 1107.5, expected MH₂²⁺: 1107.0).

### Example 13

### Synthesis of [Chelator 2A]-Cinnamycin Conjugate (Conjugate 4)

Cinnamycin (Sigma-Aldrich; 2.0 mg, 1.0 μmol), Boc-protected Chelator 2A (Example 6; 1.1 mg, 1.5 μmol) and DIPEA (0.5 μL, 2.9 μmol) were dissolved in DMF (1.0 ml). The reaction mixture was shaken overnight. The mixture was then diluted with 20% ACN/water/0.1% TFA (6 ml) and the product purified using preparative HPLC.

### Purification and Characterization.

Purification by preparative HPLC (gradient: 30-70% B over 40 min) afforded 1.8 mg pure Boc-protected Conjugate 4. The purified material was Boc-deprotected in TFA/4% water (2 ml) for 45 min and lyophilized from 50% ACN/water affording 1.6 mg Conjugate 4 (yield 73%). The material was analysed by analytical LC-MS (gradient: 10-40% B over 5 min, t_{R}: 3.7 min. found m/z: 1120.9, expected MH₂²⁺: 1121.0).

### Example 14

### Preparation of ^{99m}Tc-Labelled Chelator-Conjugates

The radiolabelled preparations were used either (i) without purification (high RCP at high RAC); or (ii) with purification to remove unlabelled LBP peptide.

Conjugate 3A (0.1 mg, 40 nmol) was dissolved in a mixture of ethanol (100 μL) and water (100 μL) and placed in a sonic bath for ˜20 min to aid solubility. The solution was added to a lyophilised kit [formulation: SnCl₂.2H₂O (0.016 mg, 0.07 μmol), MDP(H₄) (0.025 mg, 0.14 μmol), NaHCO₃ (4.5 mg, 53.6 μmol), Na₂CO₃ (0.6 mg, 5.66 μmol) and NaPABA (0.2 mg, 1.26 μmol)].

[^{99m}TcO₄]⁻ eluate (˜1 ml) from a ⁹⁹Mo/^{99m}Tc generator was then added and the mixture was left to stand for ˜10 min at room temperature. A portion of crude product (˜400 μL) was injected onto the HPLC column (see HPLC conditions below). The radioactive peak with a retention time of ca. 18 min was “cut” into a vial containing PBS (various volume depending on desired RAC) and then dried in vacuo to remove excess mobile phase.

Crude RCP=93±6% (n=13). Formulated RCP (t=0)=99±1% (n=13).

Formulated RCP (t=120 min)=97±3% (n=13).

Specific Activity=4.2±0.5 GBq/nmol (n=13).

R_{T} (^{99m}Tc-Conjugate 3A)=18 min.

^{99m}Tc-Conjugate 5 was prepared following the same procedure as for Conjugate 3A:

Crude RCP=>85% (n=12). Formulated RCP (t=0)=93±7% (n=12).

Formulated RCP (t=120 min)=91±7% (n=6).

Specific Activity=3.5±0.5 GBq/nmol (n=13).

R_{T} (^{99m}Tc-Conjugate 5)=18 min.

HPLC Conditions

| Mobile Phase A | 50 mM Ammonium Acetate | |
|---|---|---|
| Mobile Phase B | Methanol | |
| Flow Rate | 1 | ml/min |
| Loop Size | 500 | μL |
| Detection (UV) | 254 | nm |

| | |
|---|---|
| Column | Phenomenex Luna C5 100R, 5u, 150 × 4.6 mm |

Gradient System

| | |
|---|---|
| 0 min | 50% (B) |
| 5 min | 50% (B) |
| 20 min | 90% (B) |
| 23 min | 90% (B) |
| 24 min | 50% (B) |
| 26 min | 50% (B) |

The RCP of the prior art HYNIC counterpart ^{99m}Tc-[Conjugate 1] was 78-89% (crude).

The conjugates of the tetra-amine chelator (Conjugates 2A and 4) were prepared similarly, except that 0.1% TFA was used as mobile phase A in place of 50 mM ammonium acetate. The retention time of ^{99m}Tc-[Conjugate 2A] was 12.2 min, and of ^{99m}Tc-[Conjugate 4] was 12.4 min.

### Example 15

### Edman Degradation of Conjugates 2A and 2B

The use of MS-analysis techniques alone were found not to be feasible for determination of the site of conjugation of the chelator, manual Edman degradation chemistry combined with LC-MS analysis was applied.

A modified literature method was used [Xu et al; PNAS, 106, p. 19310-19315 (2009); Onisko et al, J. Am. Soc. Mass Spectrom., 18, p. 1070-1079 (2007) and Hayashi et al, J Antibiotics, 43, 1421-1430 (1990)].

The data obtained demonstrated that Conjugate 2A corresponds to the N^{α} amino conjugated isomer, whereas Conjugate 2B corresponds to the Lys² N^{ε}-amino conjugate. The data did not fit with degradation products expected for a secondary amino conjugate (Lys^{d} of Formula II), proving that this site is not reactive under the conditions used for chelate conjugation. It was noted, however, that the secondary amino group does react with phenylisothiocyanate under the more forcing coupling conditions used during the Edman degradation cycles.

### Example 16

### Affinity for Phosphatidyl Ethanolamine

A Biacore 3000 (GE Healthcare, Uppsala) was equipped with an L1 chip. Liposomes made of POPE/POPC (20% PE) were applied for the affinity study using the capture technique recommended by the manufacturer. Each run consisted of activation of the chip surface, immobilization of liposomes, binding of peptide and wash off of both liposomes and peptide (regeneration). Similar applications can be found in Frostell-Karlsson et al [Pharm. Sciences, V.94 (1), (2005)]. Thorough washing of needle, tubing and liquid handling system with running buffer was performed after each cycle.

BIACORE software: The BIACORE control software including all method instructions was applied. A method with commands was also written in the BIACORE Method Definition Language (MDL) to have full control over pre-programmed instructions. BIACORE evaluation software was applied for analysing the sensorgrams. All substances were found to be good binders to phosphatidyl ethanolamine. The K_{D} for all substances was less than 100 nM. The results are given in Table 2:

TABLE 2

| | | Conjugate | Conjugate | Conjugate |
|---|---|---|---|---|
| | Duramycin | 2A | 2B | 4 |
| k_{d} (1/s) | ~8 · 10⁻⁵ | ~7 · 10⁻⁵ | ~8 · 10⁻⁴ | ~1.5 · 10⁻⁴ |
| kₐ (1/Ms) | ~2 · 10⁴ | ~1 · 10⁴ | ~4 · 10³ | ~8 · 10³ |
| K_{D} (nM) | ~5 | ~6 | ~73 | ~16 |

### Example 17

### Tumour Uptake Studies

^{99m}Tc-[Conjugate 2A], ^{99m}Tc-[Conjugate 3A] and ^{99m}Tc-[Conjugate 5] were assessed by biodistribution in the EL4 mouse lymphoma xenograft model. Briefly, following establishment of tumour growth in C57/B16 mice, the animals were treated with either:
(i) a saline/DMSO solution; or
(ii) with chemotherapy (67 mg/kg etoposide and 100 mg/kg cyclophosphamide in 50% saline 50% DMSO).

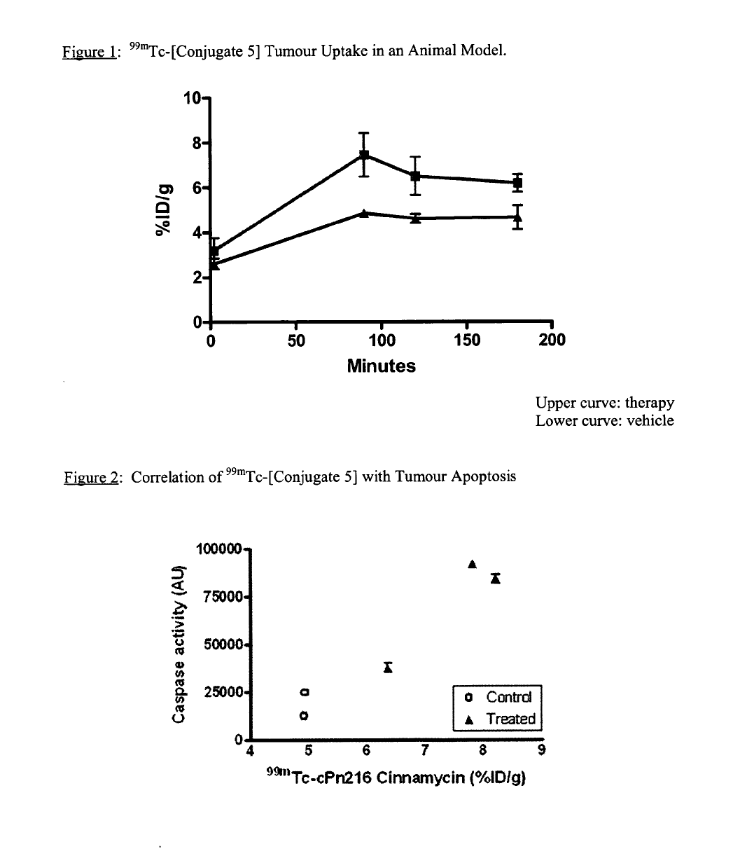

Twenty four hours after therapy or vehicle treatment, the animals were assessed for the biodistribution of the appropriate radiolabelled compound. In addition, the tumours were extracted and assessed for levels of apoptosis by measuring caspase activity (caspase-Glo assay). The correlation of binder uptake and caspase activity was then plotted for various time points. The results are shown in Table 3 (below) at 120 minutes post-injection, and in FIGS. 1 and 2 for ^{99m}Tc-[Conjugate 5]:

TABLE 3

| | | ^{99m}Tc complex of Conjugate | | |
|---|---|---|---|---|
| Ratios at 120 min p.i. | | 2A | 3A | 5 |
| Tumour:blood | vehicle | 1.3 | 1.3 | 1.2 |
| | therapy | 2 | 2 | 1.6 |
| Tumour:muscle | vehicle | 13.2 | 8 | 9.2 |
| | therapy | 20.8 | 20 | 10.8 |
| Tumour:liver | vehicle | 0.7 | 0.2 | 0.4 |
| | therapy | 1 | 0.4 | 0.8 |
| Tumour:lung | vehicle | 0.4 | 1.6 | 1.6 |
| | therapy | 0.4 | 3 | 2.8 |

### Example 18

### Of ^{99m}Tc-Labelled Chelator-Conjugates

^{99m}Tc-Conjugates were assessed by biodistribution in naïve rats to determine the pharmacokinetic profiles of the different compounds. The correlation of binder retention in different organs/tissues was then plotted for various time points. Data generated demonstrated that the inclusion of PEG in LBP1 and LBP2 conjugates improved pharmacokinetics by reducing the liver retention (see Table 4 below):

TABLE 4

| | ^{99m}Tc complex of Conjugate | | | |
|---|---|---|---|---|
| | 3A | 5 | 6 | 7 |
| Liver % ID/g | 1.01 ± 0.25 | 1.11 ± 0.05 | 0.23 ± 0.03 | 0.26 ± 0.03 |
| Urine % ID/g | 40.28 ± 2.87 | 42.07 ± 2.28 | 60.83 ± 2.14 | 58.57 ± 3.62 |

1. An imaging agent which comprises a compound of Formula I:
Z¹-(L)ₙ-[LBP]-Z² (I)
wherein:
LBP is a lantibiotic peptide of Formula II:
Cys^{a}-Xaa-Gln-Ser^{b}-Cys^{c}-Ser^{d}-Phe-Gly-Pro-Phe-Thr^{c}-Phe-Val-Cys^{b}-(HO-Asp)-Gly-Asn-Thr^{a}-Lys^{d} (II)
Xaa is Arg or Lys;
Cys^{a}-Thr^{a}, Ser^{b}-Cys^{b} and Cys^{c}-Thr^{c} are covalently linked via thioether bonds;
Ser^{d}-Lys^{d} are covalently linked via a lysinoalanine bond;
HO-Asp is β-hydroxyaspartic acid;
Z¹-(L)ₙ- is attached to the LBP Cys^{a} or when Xaa is Lys to Cys^{a} or Xaa,
wherein Z¹ comprises a ^{99m}Tc radiometal complex comprising a chelating agent having at least 4 metal donor atoms in which at least 4 of said metal donor atoms are bound to said ^{99m}Tc radiometal;
Z² is attached to the C-terminus of LBP and is OH, OB^{c}, or M^{IG},
where B^{c} is a biocompatible cation; and
M^{IG} is a metabolism inhibiting group which is a biocompatible group which inhibits or suppresses in vivo metabolism of the LBP peptide;
L is a synthetic linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1.

2. The imaging agent of claim 1, where the chelating agent is a tetradentate chelator having an N4 donor set.

3. The imaging agent of claim 2, where the N4 donor set is a diaminedioxime chelator or a tetra-amine chelator.

4. The imaging agent of claim 1, where Z¹ is attached only to Cys^{a} of LBP.

5. The imaging agent of claim 1, where Xaa is Arg.

6. The imaging agent of claim 1, where Z² is OH or OB^{c}.

7. The imaging agent of claim 1, where n is 1 and L comprises a PEG group of formula —(OCH₂CH₂)ₓ— where x is an integer of value 6 to 18.

8. A chelator conjugate of Formula III:
Z³-(L)ₙ-[LBP]-Z² (III)
wherein:
Z³ is a chelating agent having at least 4 metal donor atoms; and
L, n, LBP and Z² are as defined in claim 1.

9. A method of preparation of the imaging agent of claim 1, which comprises reaction of the chelator conjugate of claim 8 with a supply of the ^{99m}Tc radiometal in a suitable solvent.

10. A radiopharmaceutical composition which comprises the imaging agent of claim 1, together with a biocompatible carrier, in a form suitable for mammalian administration.

11. A kit for the preparation of the radiopharmaceutical composition of claim 10, which comprises the chelator conjugate of claim 8 in sterile, solid form such that upon reconstitution with a sterile supply of the ^{99m}Tc radiometal in a biocompatible carrier, dissolution occurs to give the desired radiopharmaceutical composition.

12. The kit of claim 11, where the sterile, solid form is a lyophilised solid.

13. A method of imaging the human or animal body which comprises generating an image of at least a part of said body to which the imaging agent of claim 1 has distributed using PET or SPECT, wherein said imaging agent or composition has been previously administered to said body.

14. The method of claim 13, where the part of the body is a disease state where abnormal apoptosis is involved.

15. The method of claim 13, which is carried out repeatedly to monitor the effect of treatment of a human or animal body with a drug, said imaging being effected before and after treatment with said drug, and optionally also during treatment with said drug.

16. (canceled)

17. A method of diagnosis of the human or animal body which comprises the method of imaging of claim 13.

18. The method of imaging the human or animal body of claim 13, wherein said imaging agent of claim 1 is with a biocompatible carrier and in a form suitable for mammalian administration.

19. The method of imaging the human or animal body of claim 13, wherein said imaging agent is a radiopharmaceutical composition prepared from the kit of claim 11.

20. The method of diagnosis of claim 17, further comprising generating an image of at least a part of said body wherein at least the part of the body is a disease state where abnormal apoptosis is involved.


SEQUENCE LISTING (4 sequences)

SEQ ID NO: 1 - protein, 19 aa, Streptoverticillium cinnamoneum
  MISC_FEATURE (1)..(1): chelator conjugated
CRQSCSFGPPTFVCXGNTK

SEQ ID NO: 2 - protein, 19 aa, Streptoverticillium cinnamoneum
  THIOETH (1)..(18)
  MISC_FEATURE (1)..(1): chelator conjugated
CKQSCSFGPPTFVCXGNTK

SEQ ID NO: 3 - protein, 19 aa, Streptoverticillium cinnamoneum
  THIOETH (1)..(18)
  MISC_FEATURE (1)..(1): chelator conjugated
CKQSCSFGPPTFVCXGNTK

SEQ ID NO: 4 - protein, 19 aa, Streptoverticillium cinnamoneum
  THIOETH (1)..(18)
  MISC_FEATURE (2)..(2): chelator conjugated
CKQSCSFGPPTFVCXGNTK